(19) United States
(12) Patent Application Publication
(10) Pub. No.: US 2005/0031559 A1
(43) Pub. Date: Feb. 10, 2005

(54) Cosmetic composition for caring for and/or making up skin

(75) Inventor: Guillaume Cassin, Villebon Sur Yvette (FR)

Correspondence Address: OLIFF & BERRIDGE, PLC, P.O. BOX 19928, ALEXANDRIA, VA, 22320, US

(73) Assignee: L'OREAL, PARIS (FR)

(21) Appl. No.: 10/885,141

(22) Filed: Jul. 7, 2004

Related U.S. Application Data
(60) Provisional application No. 60/497,558, filed on Aug. 26, 2003.

(30) Foreign Application Priority Data
Jul. 7, 2003 (FR) 03 08299

(51) Int. Cl. A61K 7/135; A61K 7/021

(52) U.S. Cl. 424/62; 424/63

(57) ABSTRACT

Cosmetic compositions for making up and/or caring for skin, especially the face, include, in a physiologically acceptable medium, at least porous mineral particles containing at least one optical brightener.

### CROSS-REFERENCE TO RELATED APPLICATIONS

This application claims the benefit of French Application No.03 08299 filed on Jul. 7, 2003 and U.S. Provisional Application No. 60/497,558 filed on Aug. 26, 2003, the entire disclosures of which are incorporated by reference herein.

### BACKGROUND OF THE INVENTION

The present invention relates to cosmetic compositions for caring for and/or making up skin, especially the body, hands, neck and face.

Compositions according to the invention may constitute skin makeup products, especially for the face, with lightening properties.

A certain number of people with colored or even dark skin are in search of cosmetic compositions for lightening the skin. To obtain this skin bleaching effect, cosmetic or dermatological compositions containing one or more lightening agents or bleaching agents are generally used. However, to obtain a noticeable lightening effect, these compositions must firstly comprise large amounts of lightening agents, and must secondly be used for a long time. Specifically, in most cases, no immediate lightening effect is observed directly on application of compositions comprising lightening agents.

Representative lightening agents commonly used include agents with an optical effect, and in particular optical brighteners. Such agents have the capacity to induce, under suitable conditions of use, a change in the visual appearance of a surface onto which they are applied.

EP 0 962 224 and WO 00/71085 propose the use of optical brighteners, such as stilbene derivatives, coumarin derivatives, oxazole and benzoxazole derivatives, and imidazole derivatives, as agents for lightening the skin via an optical effect. Such compounds are available, for example, under the trade names Tinopal SOP and Uvitex OB from the company Ciba-Geigy. These brighteners are dispersed directly in cosmetic formulations, which may, in certain cases, affect their physicochemical stability. Specifically, it is not possible to entirely eliminate a potential reactivity of these agents towards other components of cosmetic formulations, and vice versa.

In order to partially overcome this problem, it has been proposed to fix optical brighteners onto polymers or copolymers. The fact that an optical brightener is covalently bonded onto a polymer chain prevents it from being released into a surrounding medium, which makes the optical brightener less sensitive, or even insensitive, to chemical reactions and renders the optical brightener compatible with other dyes, if necessary.

EP 1 099 716 describes a composition for topical application comprising, in a physiologically acceptable medium, at least one copolymer comprising at least one optical brightener fixed to the copolymer chain via a covalent bond. This copolymer is formed from two types of polymer units and the optical brightener is fixed to one of them so as to represent about 4% to 30% by weight relative to the weight of the copolymer.

EP 1 191 041 describes aqueous polymer dispersions containing dyes. The dispersions of organic particles described comprise a polymer matrix consisting of ethylene-based unsaturated monomers and at least one coloring agent uniformly distributed in the polymer matrix and chosen from dyes, UV-absorbing agents and optical brighteners in a weight concentration of between 0.5% and 50% by weight, calculated relative to the polymer matrix. However, the fraction of dye present in the core of the particles cannot afford an optical effect. Consequently, the optical effect observed with particles of this type is not optimum.

It is also known practice to fix optical brighteners onto a mineral particulate support. FR 2 810 881 describes microparticles with a specific surface area of less than 100 m²/g or even less than 50 m²/g, and thus of low porosity, for instance clays or alumina, which are surface-functionalized with optical agents, especially optical brighteners. The brighteners are fixed by adsorption and/or complexing onto the surface of the porous mineral particles.

### SUMMARY OF THE INVENTION

The inventors have discovered, surprisingly, that it is possible to significantly reduce the amount of brightener generally required to obtain an intended bleaching effect, provided that brighteners are introduced in a form combined with specific mineral particles.

Specific mineral particles can be particles with a high degree of porosity, which are especially compatible with internal immobilization, for example by impregnation of brighteners.

In various exemplary embodiments, the present invention relates to compositions for making up and/or caring for skin, especially the face, comprising, in a physiologically acceptable medium, at least porous mineral particles containing at least one optical brightener.

In various exemplary embodiments, mineral particles have a specific surface area of 500 m²/g or more.

In various exemplary embodiments, the present invention relates to the use of porous mineral particles containing at least one optical brightener as a skin-lightening agent in cosmetic compositions.

In various exemplary embodiments, the present invention also relates to skin makeup processes comprising applying one or more compositions according to the invention to the skin.

In various exemplary embodiments, the present invention relates to cosmetic processes for lightening skin, especially the face and/or the neck, comprising at least one step of applying one or more compositions according to the invention to the skin.

Advantageously, exemplary compositions according to the invention can give skin onto which they are applied, and in particular as regards the complexion, improved qualities in terms of uniformity, homogeneity, transparency and whiteness. This results in a visual effect of uniform porcelain type.

Exemplary compositions according to the invention can be endowed with concealing properties.

Exemplary compositions according to the invention can attenuate skin surface defects. For example, exemplary compositions according to the invention can mask wrinkles and pores and/or camouflage colored skin defects, such as redness or marks.

Desirable properties described herein can be obtained using a reduced amount of brightener. For example, as emerges from the examples given below, a significantly reduced phenomenon of leaching of optical brightener may be observed by exemplary compositions according to this invention. For example, in an aqueous medium, leaching can be less than 0.01% of the initial concentration of optical brightener, less than 1% in an aqueous-alcoholic solvent and less than 5% in a silicone oil.

### DETAILED DESCRIPTION OF EXEMPLARY EMBODIMENTS

Porous Mineral Particles

Exemplary particles according to the invention can be of inorganic nature (e.g., mineral nature), and can have a porous character that is sufficient to at least partially allow incorporation of one or more brighteners into their structure.

Porous mineral particles according to the invention may have a specific surface area, for example, of 500 m²/g or more, 600 m²/g or more or 700 m²/g or more, assessed according to the BET method.

“BET specific surface area” is determined according to the BET (Brunauer-Emmet-Teller) method described in the “Journal of the American Chemical Society,” 60: 309 (1938) and corresponding to international standard ISO 5794/1 (appendix D). BET specific surface area corresponds to the total specific surface area (thus including micropores) of porous mineral particles under consideration.

Exemplary porous mineral particles may or may not have a spherical shape.

The number-average size of exemplary porous mineral particles may range, for example, from 0.1 to 50 μm, 0.1 to 20 μm or 0.5 to 10 μm.

The term “number-average size” refers to the dimension given by the statistical particle size distribution to half the population, known as the D50.

Porous mineral particles according to the invention may be derived from any material, provided that the material is capable of showing, on the one hand, substantial or total inertness with respect to optical brighteners under consideration and other conventional components of cosmetic formulations, and, on the other hand, a porosity that is sufficient to be compatible with internal immobilization of brighteners.

Thus, exemplary porous mineral particles may comprise at least one inorganic material chosen from silica, hydroxyapatite, sericite, mica, magnesium carbonate or hydrocarbonate, glass, ceramics, aluminium oxides of alumina type and mixed silicates, for instance aluminosilicates and mixtures thereof.

As illustrations of porous mineral particles that may be suitable for the invention, mention may be made, for example, of silica particles, for instance those sold by Asahi Glass under the name “Sunsphere H series” and by Suzuki Oil and Fat under the name “GodBalls,” hydroxyapatite particles, for instance those sold by Merck (under the reference 1051990010—particle size 15 μm) or those sold by the companies Laboratory Skin Care and Sekisui under the respective names “hydroxyzomes” (LSC), AP 20C and AP 12C (Sekisui).

Porous mineral particles charged with optical brighteners may be incorporated in variable amounts into exemplary cosmetic compositions. Generally, their content is adjusted so as to obtain a desired optical effect, i.e., a visual bleaching effect. Needless to say, their content may also be directly linked to emission power of optical brighteners they contain.

In general, porous mineral particles charged with optical brightener(s) may be present in exemplary compositions according to the invention in proportions of, for example, 0.1% to 30%, 0.5% to 20% and 1% to 15% by weight relative to the weight of a composition.

Optical Brightener

Optical brighteners are compounds that are well known to those skilled in the art. Such compounds are described in “Fluorescent Whitening Agent, Encyclopedia of Chemical Technology, Kirk-Othmer,” 4th ed., 11: 227-241 (1994).

Use of optical brighteners in cosmetics, for example as optical skin-bleaching agents, exploits the fact that brighteners are chemical compounds endowed with fluorescence properties. Such compounds may absorb light in the ultraviolet range (i.e., wavelengths of less than 400 nm) and re-emit the energy by fluorescence at wavelengths ranging between 380 and 830 nm.

In various exemplary embodiments, compositions according to this invention may include optical brighteners that absorb light essentially in the UVA range (i.e., wavelengths between 300 and 390 nm) and re-emit light having a wavelength of between about 400 and about 525 nm.

In some such embodiments, compositions according to this invention may include optical brighteners that re-emit light having a wavelength between 400 and 480 nm. Light having a wavelength between 400 and 480 nm corresponds to the blue region of the visible spectrum. Emission of blue light can contribute to visually lightening skin.

Stilbene derivatives such as, for example, polystyrylstilbenes and triazinestilbenes, coumarin derivatives such as, for example, hydroxycoumarins and aminocoumarins, oxazole, benzoxazole, imidazole, triazole and pyrazoline derivatives, pyrene derivatives and porphyrin derivatives, and mixtures thereof, are known as optical brighteners.

Such compounds are widely commercially available. They include, but are not limited to, the following derivatives:

the naphthotriazole stilbene derivative sold under the trade name “Tinopal GS,” disodium 4,4′-distyrylbiphenylsulfonate (CTFA name: disodium distyrylbiphenyl disulfonate) sold under the trade name “Tinopal CBS-X,” the cationic aminocoumarin derivative sold under the trade name “Tinopal SWN Conc.,” the sodium 4,4′-bis[(4,6-dianilino-1,3,5-triazin-2-yl)amino]stilbene-2,2′-disulfonate sold under the trade name “Tinopal SOP,” the 4,4′-bis[(4-anilino-6-bis(2-hydroxyethyl)amino-1,3,5-triazin-2-yl)amino]stilbene-2,2′-disulfonic acid sold under the trade name “Tinopal UNPA-GX,” the 4,4′-bis[anilino-6-morpholine-1,3,5-triazin-2-yl)amino]stilbene sold under the trade name “Tinopal AMS-GX,” the disodium 4,4′-bis[(4-anilino-6-(2-hydroxyethyl)methylamino-1,3,5-triazin-2-yl)amino]stilbene-2,2′-sulfonate sold under the trade name “Tinopal 5BM-GX,” all by the company Ciba Specialites Chimiques;

the 2,5-thiophenediylbis(5-tert-butyl-1,3-benzoxazole) sold under the trade name “Uvitex OB” by the company Ciba;

the anionic derivative of diaminostilbene as a dispersion in water, sold under the trade name “Leucophor BSB liquid” by the company Clariant; and

mixtures thereof.

Exemplary optical brighteners that may be used in the present invention may also be in the form of copolymers, for example acrylates and/or methacrylates, grafted with optical brightener groups as described in FR 99/10942.

Disodium 4,4′-distyrylbiphenyl sulfonate, sodium 4,4′-bis[(4,6-dianilino-1,3,5-triazin-2-yl)amino]stilbene-2,2′-disulfonate, and 2,5-thiophenediylbis(5-tert-butyl-1,3-benzoxazole) and mixtures thereof are suitable for the invention.

In various exemplary embodiments, one or more optical brighteners may be present inside porous mineral particles. In some such embodiments, some, a predominant part, all or substantially all of the one or more optical brighteners may be present inside porous mineral particles. In embodiments where all or substantially all of the one or more optical brighteners may be present inside porous mineral particles, none or substantially none of the one or more optical brighteners are immobilized at the surface of the particles. In contrast to known compositions including brighteners, compositions according to the present invention including particles and one or more optical brighteners do not involve, or at the very least not mainly, immobilization of the one or more optical brighteners at the surface of particles.

In various exemplary embodiments, optical brighteners are immobilized by impregnation into porous mineral particles.

Combinations of at least one optical brightener with porous mineral particles may, for example, be obtained via a preparation process comprising:

mixing at least one optical brightener in an organic solvent with porous mineral particles;

stirring the mixture at room temperature until the brightener has fully dissolved;

evaporating off the organic solvent; and

recovering the porous mineral particles combined with the optical brightener.

Porous particles can be in the form of a powder including porous mineral particles impregnated with optical brighteners or whose pores are filled with optical brighteners in solid form.

The amount of optical brightener associated with particles depends on the desired effect. However, optical brighteners are generally present in porous mineral particles in an amount of active material ranging from, for example, 0.5% to 50%, 0.75% to 40% and 1% to 35% by weight relative to the total weight of particles.

Physiologically Acceptable Medium

Exemplary compositions according to the invention may comprise a physiologically acceptable medium.

The term “physiologically acceptable medium” refers to a non-toxic medium that may be applied to human skin or lips. Physiologically acceptable media are generally adapted to the nature of the support onto which a composition is to be applied and also to an appearance that a composition is intended to condition.

Exemplary compositions according to the invention may be more or less fluid and may, for example, have the appearance of a white or colored cream, an ointment, a milk, a lotion, a serum, a paste or a mousse. Exemplary compositions may also be in solid form, such as in the form of a stick.

When exemplary compositions of the invention are emulsions, the proportion of a fatty phase may, in various exemplary embodiments, range from 5% to 80% relative to the total weight of a composition. In some such embodiments, the proportion of a fatty phase may range from 5% to 50% by weight relative to the total weight of a composition.

Oils, emulsifiers and co-emulsifiers used in exemplary compositions in emulsion form are chosen from those conventionally used in the field under consideration.

Emulsifiers and co-emulsifiers are generally present in exemplary compositions in a proportion ranging from 0.3% to 30% by weight and preferably from 0.5% to 20% by weight relative to the total weight of a composition.

In various exemplary embodiments, compositions in emulsion form may contain no emulsifier.

Exemplary compositions of the invention may comprise an oil, chosen, for example, from:

hydrocarbon-based oils of animal origin, such as perhydrosqualene;

hydrocarbon-based oils of plant origin, such as liquid triglycerides of fatty acids containing from 4 to 10 carbon atoms and the liquid fraction of karite butter;

synthetic esters and synthetic ethers, for example, of fatty acids, for instance oils of formulae R¹COOR² and R¹OR² in which R¹ represents a fatty acid residue containing from 8 to 29 carbon atoms and R² represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;

linear or branched hydrocarbons of mineral or synthetic origin, such as volatile or non-volatile liquid paraffins, and derivatives thereof, petroleum jelly, polydecenes, and hydrogenated polyisobutene such as parleam oil;

fatty alcohols containing from 8 to 26 carbon atoms, for instance cetyl alcohol, stearyl alcohol and mixtures thereof (cetylstearyl alcohol), octyldodecanol, 2-butyloctanol, 2-hexyldecanol, 2-undecylpentadecanol, oleyl alcohol or linoleyl alcohol;

partially hydrocarbon-based and/or silicone-based fluoro oils, for instance those described in document JP-A-2 295 912;

silicone oils, for instance volatile or non-volatile polymethylsiloxanes (PDMSs) containing a linear or cyclic silicone chain, that are liquid or pasty at room temperature such, for example, cyclopolydimethylsiloxanes (cyclomethicones) such as cyclohexasiloxane; polydimethylsiloxanes comprising alkyl, alkoxy or phenyl groups, that are pendent or at the end of a silicone chain, these groups containing from 2 to 24 carbon atoms; phenylsilicones, for instance phenyltrimethicones, phenyldimethicones, phenyltri-methylsiloxydiphenylsiloxanes, diphenyldimethicones, diphenylmethyldiphenyltrisiloxanes, 2-phenylethyltrimethylsiloxysilicates and polymethylphenylsiloxanes; and

mixtures thereof.

For use in cosmetic treatment of greasy or combination skin, exemplary compositions according to the invention may be, for example, in the form of oil-in-water (O/W) emulsions having outer aqueous phases that provide a freshness effect.

Exemplary compositions according to the invention may also comprise at least one wax, at least one gum and/or at least one pasty fatty substance, of plant, animal, mineral or synthetic origin, of silicone or non-silicone nature.

Waxes may be hydrocarbon-based waxes, silicone waxes and/or fluoro waxes, optionally comprising ester or hydroxyl functions. Waxes may, for example, be of natural origin.

Waxes may represent, for example, from 0.01% to 10% or 0.1% to 5% by weight relative to the total weight of a composition. In various exemplary embodiments, compositions may be wax-free.

Compositions according to the invention may also contain adjuvants that are common in cosmetics, such as hydrophilic or lipophilic gelling agents, dyestuffs, hydrophilic or lipophilic active agents, preserving agents, moisturizers, sequestering agents, antioxidants, solvents, fragrances, fillers, physical sunblocks and chemical sunscreens, odor absorbers and pH regulators (acids or bases), and mixtures thereof.

Amounts of various adjuvants are those conventionally used in the field under consideration, for example from 0.01% to 20% of the total weight of a composition. Depending on their nature, adjuvants may be introduced into a fatty phase or into an aqueous phase. In any case, adjuvants, and the proportions thereof, will, preferably, be chosen so as not to harm desired properties according to various exemplary embodiments of the invention.

As emulsifiers and co-emulsifiers that may be used in the invention, mention may be made, for example, of O/W emulsifiers such as fatty acid esters of polyethylene glycol such as, for example, PEG-100 stearate and fatty acid esters of glycerol, such as glyceryl stearate, and also water-in-oil (W/O) emulsifiers such as the oxyethylenated poly(methylcetyl)(dimethyl)methylsiloxane sold under the trade name Abil WE09 from the company Degussa Goldschmidt or mixtures of acetyl ethylene glycol stearate and glyceryl tristearate sold, for example, by the company Guardian under the trade name Unitwix.

Hydrophilic gelling agents that may be mentioned include, but are not limited to, carboxyvinyl polymers (carbomer), acrylic copolymers such as acrylate/alkylacrylate copolymers, polyacrylamides, polysaccharides, natural gums and clays, and lipophilic gelling agents that may be mentioned include, but are not limited to, modified clays, for instance bentones, metal salts of fatty acids, hydrophobic silica and polyethylenes.

As examples of dyestuffs that may be used according to the invention, mention may be made of lipophilic dyes, hydrophilic dyes, pigments and nacres usually used in cosmetic or dermatological compositions and mixtures thereof.

Dyestuffs can be incorporated into compositions of the invention in amounts adjusted so as not to substantially adversely affect the desired optical effect by the presence of the optical brighteners.

To do this, dyestuffs are generally present in a proportion of, for example 0.01% to 10%, 0.05% to 7%, 0.1% to 7% and 0.1% to 5% by weight relative to the total weight of a composition.

Exemplary liposoluble dyes include, for example, Sudan Red, DC Red 17, DC Green 6, β-carotene, soybean oil, Sudan Brown, DC Yellow 11, DC Violet 2, DC Orange 5 and quinoline yellow.

Exemplary pigments may be white or colored, mineral and/or organic and coated or uncoated. Among mineral pigments that may be mentioned are titanium dioxide, optionally surface-treated, zirconium oxide or cerium oxide, and also iron oxide or chromium oxide, manganese violet, ultramarine blue, chromium hydrate and ferric blue. Among organic pigments that may be mentioned are carbon black, pigments of D&C type and lakes based on cochineal carmine or on barium, strontium, calcium or aluminium.

Exemplary nacreous pigments may be chosen from white nacreous pigments such as mica coated with titanium or with bismuth oxychloride, colored nacreous pigments such as titanium mica with iron oxides, titanium mica with, for example, ferric blue or chromium oxide, titanium mica with an organic pigment of the abovementioned type, and nacreous pigments based on bismuth oxychloride.

In various exemplary embodiments, pigments may be subjected to a surface treatment.

As fillers that may be used in exemplary compositions of the invention, mention may be made, for example, besides pigments, of silica powder; talc; starch crosslinked with octenylsuccinic anhydride sold by the company National Starch under the name Dry Flo Plus (28-1160); polyamide particles such as, for example, those sold under the name Orgasol by the company Atochem; polyethylene powders; microspheres based on acrylic copolymers, such as those made of ethylene glycol dimethacrylate/lauryl methacrylate copolymer sold by the company Dow Coming under the name Polytrap; expanded powders such as hollow microspheres and, for example, the microspheres sold under the name Expancel by the company Kemanord Plast or under the name Micropearl F 80 ED by the company Matsumoto; silicone resin microbeads such as those sold under the name Tospearl by the company Toshiba Silicone; and mixtures thereof.

Fillers may be present in amounts ranging from 0 to 20% by weight and preferably from 1% to 10% by weight, relative to the total weight of a composition.

Fillers that are advantageous for use in the present invention include, but are not limited to, silica, mica and titanium dioxide.

As active agents that are common in cosmetics or dermatology, which may be used according to the invention, mention may be made, for example, of any active agent known for its activity on ageing of the skin, for instance keratolytic agents or pro-desquamating agents, for example α-hydroxy acids, β-hydroxy acids, α-keto acids, β-keto acids, retinoids and esters thereof, retinal, and retinoic acid and its derivatives. Mention may also be made of venotonic plant extracts such as extracts of ruscus and/or of common horse chestnut; xanthine bases such as caffeine; vitamins, for instance vitamins A, B3, PP, B5, E, K1 and/or C and derivatives of these vitamins and, for example, esters thereof; free-radical scavengers; sunscreens; moisturizers, for instance polyols; ceramides; DHEA and its derivatives; coenzyme Q10; bleaching agents and depigmenting agents functioning via biological action, for instance kojic acid, extracts of skullcap, of mulberry, of liquorice and/or of camomile; para-aminophenol derivatives, arbutin and derivatives thereof and mixtures thereof.

For use in cosmetic treatment of greasy or combination skin, exemplary compositions according to the invention may contain, for example, at least one active agent chosen from: vitamins B3 and B5; zinc salts such as, for example, zinc oxide and zinc gluconate; salicylic acid and its derivatives, such as 5-n-octanoylsalicylic acid; triclosan; capryloylglycine; an extract of clove; octopirox; hexamidine; and azelaic acid and its derivatives.

In the event of incompatibility or to stabilize them, at least some of the active agents mentioned above may be incorporated into spherules such as, for example, ionic or nonionic vesicles and/or nanoparticles (nanocapsules and/or nanospheres).

UVA and/or UVB screening agents chosen from organic screening agents and mineral screening agents that are optionally coated to make them hydrophobic may also be introduced into exemplary compositions according to the invention.

Exemplary compositions according to the invention may be, for example, in any galenical form normally used in cosmetics, and may, for example, be in the form of a solution such as, for example, an optionally gelled oily solution, an emulsion of liquid or semi-liquid consistency of the milk type, obtained by dispersing a fatty phase in an aqueous phase (O/W) or, conversely, (W/O), a triple emulsion (W/O/W or O/W/O) or a suspension or emulsion of soft, semi-solid or solid consistency of the cream or gel type, or alternatively microemulsions, microcapsules, microparticles or a vesicular dispersion of ionic type (liposomes or oleosomes) and/or of nonionic type (niosomes) and/or a dispersion of nanocapsules or nanospheres.

Exemplary compositions according to the invention may be in the form of makeup products for the face.

This invention is illustrated by the following Examples, which are merely for the purpose of illustration.

### Example 1

Preparation of porous mineral particles containing an optical brightener.

A solution containing 1 gram of optical brightener (Uvitex OB), 230 ml of acetone and 2 grams of porous silica (Sunsphere H51 sold by the company Asahi Glass) is prepared. This solution is stirred at room temperature until the brightener has fully dissolved. The solution is then transferred into a round-bottomed flask and the acetone is evaporated off on a rotavapor at 40° C. After total evaporation of the solvent, a powder consisting of porous silica particles whose pores are filled with optical brightener in solid form is thus obtained. The weight composition of the particles obtained is 33% optical brightener and 66% silica.

Cosmetic compositions containing the particles according to the particular embodiment of the invention, described above, are formulated.

### Example 2

A concealer gel is prepared from the particles obtained in Example 1. Composition of the gel is indicated in Table 1 below, which also presents the composition of a control gel, i.e., a gel incorporating the optical brightener in unmodified form, that is to say not contained in particles.

TABLE 1

| Concealer gel: | Example 1 | Control |
|---|---|---|
| Ammonium polyacryldimethyltauramide | 1.00% | 1.00% |
| Uvitex OB | — | 1.33% |
| Particles according to Example 1 (33% | 4.00% | — |
| Uvitex OB-66% silica) | | |
| Water | qs 100% | qs 100% |

Evaluation of the Optical Efficacy

The optical efficacies obtained for the composition according to the invention and the control composition containing an equivalent amount of optical brightener are measured.

The measurement is performed in the following manner: a film of the composition is spread on a contrast card using a film spreader (film thickness equal to 50 μm). The film is left to dry and the spectral reflectance with and without UV is then measured using a spectroreflectometer. The calorimetric parameters L*, a* and b* of the treated surface are determined from the reflection spectra obtained with and without UV. The total color difference between the reflectance measured without and with UV, ΔE*, is expressed by
ΔE*=[ΔL*² +Δa*² +Δb*²]^{1/2}.

The value of the total color difference ΔE* is proportionately greater the greater the brightening effect. The ΔE* values obtained are given in Table 2.

TABLE 2

| Composition | Example 1 | Control example |
|---|---|---|
| ΔE* | 1.52 | 0.30 |

These in vitro results show that, with a concentration of 4% by weight of particles containing an optical brightener (composition equivalent to 1.33% of Uvitex OB), a brightening effect that is markedly superior to that obtained with the comparative formula containing the same amount of brightener is obtained.

Leaching Test

The leaching or delayed release in various common cosmetic solvents for the optical brightener introduced into the particles according to the particular embodiment of the invention is evaluated. The compositions of the solvents used are as follows:

Solvent A: demineralized water

Solvent B: 20% demineralized water, 50% propylene glycol, 30% ethanol

Solvent C: volatile silicone oil (DC 246—Dow Corning)

A defined amount of powder according to the preferred embodiment of the invention is suspended in the various solvents A, B and C. These suspensions are maintained at room temperature and stirred for 1 hour, 2 hours and 6 hours. At these various time intervals, 10 milliliters of solution are taken and are then filtered using a Sartorius Minisart 0.2 μm filter in order to remove all presence of particles. The amount of brightener released into the solution is estimated by measuring the optical density (OD) of the solution, which is related to the concentration by Beer-Lambert's law.

Beer-Lambert's law relates the optical density (OD) measured at a wavelength λ of a solution of given concentration of an absorbing molecule to the molar extinction coefficient (ε) of this molecule at the wavelength λ, to the optical path and the concentration of this molecule in the solution:
OD_{λ}=ε_{λ}1C

A reference solution with a titre of 2.3×10⁻⁵ mol.l⁻¹ makes it possible to estimate the molar extinction coefficient of the Uvitex OB at a wavelength corresponding to the absorption maximum in UV, i.e., 372 nm. The optical density of the various solutions collected at 1 hour, 2 hours and 6 hours is measured at this wavelength and the concentration of brightener released into these solutions is determined from equation 1. The percentage of leaching of the brightener into the solutions is then given by:
n %=100.Cₜ/Cᵢ

in which Ci and Ct are, respectively, the initial concentration of optical brightener and the measured concentration of optical brightener leached into the solution after a time t.

The initial concentration of optical brightener introduced into the solutions studied is 9.65×10⁻³ mol.l⁻¹ and the molar extinction coefficient of the Uvitex OB at a wavelength of 372 nm is 46 513 cm⁻¹.mol⁻¹.l.

The results obtained are given in Table 3 below.

TABLE 3

| | n % in Solvent A | N % in solvent B | n % in solvent C |
|---|---|---|---|
| 1 hour | 0.005 | 0.55 | 3.42 |
| 2 hours | 0.006 | 0.55 | 3.73 |
| 6 hours | 0.007 | 0.56 | 3.60 |

The leaching of the optical brightener is less than 0.01% of the initial concentration of optical brightener in water (solvent A), less than 1% in an aqueous-alcoholic solvent (solvent B) and less than 5% in a silicone oil (solvent C).

While this invention has been described in conjunction with the exemplary embodiments and examples outlined above, various alternatives, modifications, variations, improvements and/or substantial equivalents, whether known or that are or may be presently unforeseen, may become apparent to those having at least ordinary skill in the art. Accordingly, the exemplary embodiments of the invention, as set forth above, are intended to be illustrative, not limiting. Various changes may be made without departing from the spirit and scope of the invention. Therefore, the invention is intended to embrace all known or later developed alternatives, modifications, variations, improvements and/or substantial equivalents.

1. A cosmetic composition for making up and/or caring for skin, comprising porous mineral particles containing at least one optical brightener, wherein the porous mineral particles are provided in a physiologically acceptable medium.

2. The cosmetic composition according to claim 1, wherein the porous mineral particles have a BET specific surface area of 500 m²/g or more.

3. The cosmetic composition according to claim 1, wherein the porous mineral particles have a BET specific surface area of 600 m²/g or more.

4. The cosmetic composition according to claim 1, wherein the porous mineral particles have a BET specific surface area of 700 m 2/g or more.

5. The cosmetic composition according to claim 1, wherein the porous mineral particles have a number-average size ranging from 0.1 to 50 μm.

6. The cosmetic composition according to claim 1, wherein the porous mineral particles have a number-average size ranging from 0.1 to 20 μm.

7. The cosmetic composition according to claim 1, wherein the porous mineral particles have a number-average size ranging from 0.5 to 10 μm.

8. The cosmetic composition according to claim 1, wherein the porous mineral particles comprise at least one particle selected from the group consisting of silica, hydroxyapatite, sericite, glass, ceramic, mica, magnesium carbonate, magnesium hydrocarbonate, magnesium oxides of alumina type, mixed silicates and mixtures thereof.

9. The cosmetic composition according to claim 8, wherein the mixed silicates are aluminosilicates.

10. The cosmetic composition according to claim 1, wherein the porous mineral particles are selected from the group consisting of silica particles and hydroxyapatite particles.

11. The cosmetic composition according to claim 1, comprising porous mineral particles charged with the at least one optical brightener in a proportion of from 0.1% to 30% by weight relative to a total weight of the composition.

12. The cosmetic composition according to claim 1, comprising porous mineral particles charged with the at least one optical brightener in a proportion of from 0.5% to 20% by weight relative to a total weight of the composition.

13. The cosmetic composition according to claim 1, comprising porous mineral particles charged with the at least one optical brightener in a proportion of from 1% to 15% by weight relative to a total weight of the composition.

14. The cosmetic composition according to claim 1, wherein the at least one optical brightener is immobilized in the porous mineral particles.

15. The cosmetic composition according to claim 1, wherein the at least one optical brightener is present in an active material content ranging from 0.5% to 50% by weight relative to a total weight of the porous mineral particles.

16. The cosmetic composition according to claim 1, wherein the at least one optical brightener is selected from the group consisting of stilbene derivatives; coumarin derivatives; oxazole, benzoxazole, imidazole, triazole and pyrazoline derivatives; pyrene derivatives; porphyrin derivatives; and mixtures thereof.

17. The cosmetic composition according to claim 1, wherein the at least one optical brightener is selected from the group consisting of:
naphthotriazole stilbene derivatives, disodium 4,4′-distyrylbiphenylsulfonate (CTFA name: disodium distyrylbiphenyl disulfonate), sodium 4,4′-bis[(4,6-dianilino-1,3,5-triazin-2-yl)amino]stilbene-2,2′-disulfonate, 4,4′-bis[(4-anilino-6-bis(2-hydroxyethyl)amino-1,3,5-triazin-2-yl)amino]stilbene-2,2′-disulfonic acid, 4,4′-bis[anilino-6-morpholine-1,3,5-triazin-2-yl)amino]stilbene, disodium 4,4′-bis[(4-anilino-6-(2-hydroxyethyl)methylamino-1,3,5-triazin-2-yl)amino]stilbene-2,2′-sulfonate;
2,5-thiophenediylbis(5-tert-butyl-1,3-benzoxazole);
an anionic diaminostilbene derivative as a dispersion in water; and
mixtures thereof.

18. The cosmetic composition according to claim 1, wherein the at least one optical brightener is selected from the group consisting of disodium 4,4′-distyrylbiphenyl-sulfonate, sodium 4,4′-bis[(4,6-dianilino-1,3,5-triazin-2-yl)amino]stilbene-2,2′-disulfonate, 2,5-thiophenediylbis(5-tert-butyl-1,3-benzoxazole) and mixtures thereof.

19. The cosmetic composition according to claim 1, further comprising at least one compound selected from the group consisting of hydrophilic dyes, lipophilic dyes, pigments, fragrances, preserving agents, physical sunblocks, chemical sunscreens, sequestering agents, liposoluble active agents, water-soluble active agents, fillers, moisturizers, pH regulators and mixtures thereof.

20. The cosmetic composition according to claim 1, wherein the composition is in a form selected from the group consisting of: a solution, a gel, a lotion-type dispersion, a serum-type dispersion, an emulsion of liquid or semi-liquid consistency of milk type obtained by dispersing a fatty phase in an aqueous phase (O/W), an emulsion of liquid or semi-liquid consistency of milk type obtained by dispersing an aqueous phase in a fatty phase (W/O), a suspension of soft, semi-solid or solid consistency of the cream or gel type, an emulsion of soft, semi-solid or solid consistency of the cream or gel type, microemulsions, microcapsules, microparticles, vesicular dispersions of ionic type and vesicular dispersions of nonionic type.

21. The cosmetic composition according to claim 1, wherein the composition is in a form of a makeup product for the face.

22. A method of forming a skin-lightening composition comprising incorporating porous mineral particles containing at least one optical brightener into the composition.

23. A process for making up the skin, comprising applying the cosmetic composition according to claim 1 to the skin.

24. A cosmetic process for lightening the skin, comprising applying the cosmetic composition according to claim 1 to the skin.
